(19) United States
(12) Patent Application Publication
(10) Pub. No.: US 2012/0308998 A1
(43) Pub. Date: Dec. 6, 2012

(54) METHODS AND SYSTEMS FOR REDUCING DNA FRAGMENTATION IN A POPULATION OF SPERM CELLS

(75) Inventors: Juan Moreno, College Station, TX (US); Michael Evans, College Station, TX (US); Michael Kjelland, Valley City, ND (US); Jaime Gosalvez, Colmenar Viejo (ES); Carmen Lopez-Fernandez, Colmenar Viejo (ES)

(73) Assignee: INGURAN, LLC, Navasota, TX (US)

(21) Appl. No.: 13/519,114

(22) PCT Filed: Oct. 28, 2010
(86) PCT No.: PCT/US2010/054549

§ 371 Date: Jun. 25, 2012

Related U.S. Application Data
(60) Provisional application No. 61/256,893, filed on Oct. 30, 2009.

(51) Int. Cl. G01N 21/64; A61B 17/43; C12M 1/34; C12Q 1/68; G01N 21/17

(52) U.S. Cl. 435/6.1; 435/287.2; 600/35

(57) ABSTRACT

A method and system for sorting sperm samples according to different levels of DNA fragmentation and methods of using populations with low levels of DNA fragmentation to improve fertility and success rates of assisted reproductive procedures, including artificial insemination, in vitro fertilization, intracytoplasmic injection, and other related techniques.

### FIELD

The present embodiments generally relate to methods and systems for reducing the DNA fragmentation in a population of sperm, and more particularly, to generating sperm populations with reduced DNA fragmentation for use in assisted reproductive technology for the production of offspring.

### BACKGROUND

Sperm and sex sorted sperm (sperm sorted based on carrying an X or Y chromosome) are biological materials of great interest for assisted reproduction and in the livestock breeding industry. However, damaged and/or dead sperm lack the viability for producing offspring through artificial insemination (AI), in vitro fertilization (IVF), Intracytoplasmic Sperm Injection (ICSI) or other assisted reproductive technologies. Damaged cells can include cells with altered membranes, cells undergoing apoptosis as well as cells with DNA fragmentation. A damaged sperm, when present in a viable sperm population used in an assisted reproductive technology, may be capable of fertilizing an egg, but may fail to produce a viable embryo or may produce an embryo having genetic abnormalities that will not develop properly or may die later. In this way, sperm with DNA fragmentation competes with viable sperm and reduces the overall likelihood of a successful pregnancy and increases the likelihood of producing malformed offspring. Simon et al. Human Reproduction, Vol. 25 No. 7 pp. 1594-1608 (2010) demonstrate the negative impact of increased rates of sperm DNA fragmentation on pregnancy rates and embryonic development following assisted reproductive technology, such as in IVF and ICSI. Likewise, an egg, oocyte, embryo or other reproductive cell with damaged DNA or DNA fragmentation reduces the chances of a successful pregnancy. Therefore, a need exists for methods and systems relating to sperm processing which reduce the levels of DNA fragmentation in a sorted sperm subpopulation and particularly in sorted subpopulations used in assisted reproductive technology where sex selection is the final target.

Sex sorted sperm are gender enriched populations of sperm characterized as sperm sorted on the basis of carrying an X chromosome or carrying a Y chromosome. The use of sex sorted sperm can particularly benefit the dairy and beef industries by providing offspring of the desired gender with a high degree of certainty. Flow cytometry, as applied to sperm for sex sorting, generally incorporates a non toxic DNA binding fluorescent dye which permeates the cell membrane and associates with the DNA of each sperm in a stoichiometric manner. The volume of the dye associated with each sperm is closely related to the amount of DNA contained within each cell, and laser excitation causes distinguishable fluorescence in sperm bearing Y-chromosomes and bearing X-chromosomes. U.S. Pat. Nos. 5,135,759, 6,357,307, 7,371,517 and 7,758,811, each of which are incorporated herein by reference, provide systems and methods adapted to sorting sperm based on the differences in the X and Y chromosomes.

Although fresh ejaculates of all animals inherently contain a certain baseline of sperm DNA fragmentation, the level of DNA fragmentation in ejaculates from various individual donors can vary because of different factors such as oxidative stress, apoptosis, failures in the histone-protamine replacement and other environmental factors associated with semen production. Some DNA damaging factors can be compounded during subsequent sperm processing. In addition to this and given that sperm are generally delicate cells, sperm samples, after ejaculation that are handled ex vivo, can suffer additional iatrogenic damage throughout most sperm processes while preparing the sample for insemination. In particular, the methodology of sex sorting sperm includes several steps that produce stresses on the cells that are not only damaging, but may contribute to and intensify potential iatrogenic damage. Since the damage created in certain steps of the sorting process can be compounded by the chemical and physical endured throughout the sorting process, there is a particular need to enrich the sperm cell population with respect to DNA fragmentation or the lack thereof. Therefore, a need exists for methods and systems to enrich sperm samples taking into account the levels of DNA fragmentation for improving the success rate of artificial reproductive technologies and the development of healthy offspring using sex sorted sperm.

### SUMMARY OF THE INVENTION

The present embodiments generally relate to flow cytometry methods and systems for sorting sperm and producing sperm populations exhibiting reduced amounts of DNA fragmentation as compared to the original sperm samples. These sperm populations with reduced amounts of DNA fragmentation are advantageous for insemination and/or fertilization through reproductive techniques, such as AI, ICSI, IVF and other related techniques.

Accordingly, embodiments disclosed herein provide methods and apparatus for decreasing the level of sperm DNA fragmentation in a sperm sample compared to the original sperm sample after ejaculation.

In one aspect, embodiments disclosed herein provide a method and apparatus for reducing DNA fragmentation in a sperm population.

In another aspect, embodiments disclosed herein provide a method and system for sorting sperm into populations having different DNA fragmentation characteristics including at least one sperm population enriched to have a reduced amount of DNA fragmentation.

In still another aspect, sperm are sorted on the basis of DNA fragmentation characteristics, or characteristics representative of a population of sperm which have a higher incidence of DNA fragmentation.

### BRIEF DESCRIPTION OF THE DRAWINGS

FIG. 1A illustrates a plotted representation of forward angle fluorescence versus side angle fluorescence of sperm in a flow cytometer.

FIG. 1B illustrates a side of containing sperm in a chromatin dispersion test, where the sperm were collected from a particular sort region.

FIG. 1C illustrates a side of containing sperm in a chromatin dispersion test, where the sperm were collected from a particular sort region.

FIG. 2A illustrates a plotted representation of forward angle fluorescence versus side angle fluorescence of sperm in a flow cytometer.

FIG. 2B illustrates a gated portion of sperm plotted as forward fluorescence versus integrated forward fluorescence in a flow cytometer.

FIG. 3A illustrates a graphical representation of DNA fragmentation over time in conventional sperm samples.

FIG. 3B illustrates a graphical representation of DNA fragmentation over time in sex sorted sperm samples.

FIG. 3C illustrates a graphical representation of the mean DNA fragmentation over time in conventional samples and sex sorted sperm samples.

FIG. 4 illustrates a flow cytometer in accordance with certain embodiments presented herein.

FIG. 5 illustrates a flow chart of a method in accordance with an embodiment of the present invention.

### DETAILED DESCRIPTION OF THE PREFERRED EMBODIMENTS

Before explaining the method in detail it is to be understood that the method is not limited to the particular embodiments described herein and can be carried out in a variety of ways. Furthermore, the methods and systems described are disclosed in a general fashion, so they may be applied to specific systems once the general principals are understood.

One embodiment relates to a method for sorting cells which generate discrete subpopulations where some subpopulations are enriched with cells having reduced levels or even no DNA fragmentation and other subpopulations enriched with cells having increased levels of DNA fragmentation. The sorting can be based on the presence of DNA fragmentation characteristics to reduce the incidence of DNA fragmentation in an enriched sperm sample. The method can begin by establishing a sperm sample. The sperm sample can be acquired from any mammal without limitation, including those listed by Wilson, D. E. and Reeder, D. M., Mammal Species of the World, Smithsonian Institute Press (1993), the entire text of which is incorporated herein by reference. In one embodiment, neat semen can be processed by dilution with extenders known in the art for preserving the motility and fertility of sperm. In another embodiment, the sperm sample can also be established by thawing previously cryopreserved and/or previously processed sperm from straws. In yet another embodiment, the sperm sample can comprise sperm heads removed from their respective sperm tails.

In some embodiments, the sperm sample can be stained with a marker or dye. The marker can be a DNA selective or DNA binding dye, such as Hoechst 33342, Hoechst 33258, BBC, SYBR-14, SYBR Green I, a bisbenzimide dye, or a combination thereof, which may be a fluorochrome. In some embodiments, the sperm can be dyed with a second marker, such as a red food dye or propidium iodide.

In such embodiments, the stained sperm can be individually evaluated using flow cytometry, or another analytical technique based on fluorescent or visible microscopy. In flow cytometry sperm are entrained within a fluid stream then broken off droplets, each droplet ideally containing a single sperm which is individually irradiated with an energy source, such as a laser, at an inspection zone. The DNA selective fluorochrome dye will absorb the energy of the laser and emit light at a different wavelength in response to the excitation. The amount of this emission can be quantified to determine the relative amount of dye as compared to other sperm in the sample.

The emission of the irradiated sperm can then be detected or monitored through the use of a photomultiplier tube that produces a signal representative of the intensity of the emission from the irradiated cells. The intensity of the emission can provide information regarding the ability of each sperm to differentially interact with the different dyes used and can provide information regarding DNA fragmentation characteristics of the sperm. Because cells having larger amounts of DNA fragmentation may have fewer binding sites for a DNA selective dye, such a cells might produce less intense emissions allowing for the evaluation of DNA fragmentation characteristics based upon the detected emission values. DNA fragmentation characteristics can include other distinguishable cellular properties such as compromised or altered membranes, in which case a quencher or second marker can greatly reduce the amount an excited sperm cell fluoresces. Based upon the evaluation of one or more of these characteristics, a subpopulation of cells can be characterized as containing a higher or lower level of DNA fragmentation, or a higher or lower likelihood of having such damage, than a as compared to the remainder of the population of sperm in a sample. By selecting a population of sperm characterized as less likely to have DNA fragmentation, an enriched population with lower levels of DNA fragmentation can be sorted and collected.

For example, in some sperm sorting systems using flow cytometry, sperm can be sorted by electrically charging the stream entraining the sperm based upon the produced signal. The charged droplets that form and depart from the fluid stream then retain that charge and can be electromagnetically deflected by deflection plates guiding the droplet into one of several containers.

Such a flow cytometer system can be configured to sort a sperm sample into a first population, a second population and possibly a third population, wherein the first population may contain a greater percentage of dead cells or cells with damaged DNA, while the second population and the third population might contain X/Y sorted products. The first population of sperm, having a higher incident of DNA fragmentation, can be collected as waste and can be included in a population considered as non-viable sperm. The remaining second and third populations can include X/Y separated populations with reduced sperm damage.

A flow cytometer system for sorting cells based on the level of DNA fragmentation characteristics can include the basic components of a flow cytometer unit including, an inlet for receiving a cell or sperm sample and an outlet in communication with an oscillator for producing droplets entraining sperm. The sperm can have a DNA selective dye associated with their DNA.

An excitation device for producing electromagnetic radiation can be used to excite the dye associated with the DNA of the sperm. A laser is one example of an excitation device, but arc lamps and other sources of radiant energy can be used for irradiating or exciting fluorochrome stained sperm.

A system for sorting sperm can include a detector positioned to detect the interaction of the excitation device with the marker associated with the DNA at the inspection zone and to produce a signal based upon the emission from the irradiated sperm. This signal can then be communicated to an analyzer for evaluating the signal produced by the detector. The signal can be evaluated for the presence of DNA fragmentation characteristics in individual sperm in the sample. DNA fragmentation characteristics can be those characteristics which indicate an increased likelihood that a subpopulation of sperm has fragmented DNA. By way of a non-limiting example, DNA fragmentation characteristics can be represented by the intensity of fluorescence emissions because of the reduced number of fluorescent dye binding cites, as well as by a reduced fluorescence caused by a second quenching dye which only associates with sperm having compromised membranes. Once a determination is made by the analyzer, a signal can be passed to a separator for separating the sample into distinct populations. One or more of the populations can be sorted into an enriched sperm sample having either higher or lower levels of DNA fragmentation as compared to the original sample.

The system can include a first collection element for collecting a first population of sorted sperm and second collection element for collecting a second population of sperm. The system can additionally include a third, or more, collection elements for collecting a third, or more, populations of sperm. Such sorting could be done simultaneously, or could be done sequentially in two or more steps. It should be appreciated that any one of the populations may be sorted solely based upon DNA fragmentation, while the other two populations can be sorted on the basis of carrying X or Y chromosomes.

### DNA Fragmentation in Sorted Sperm.

Four experiments provided herein demonstrate sorting techniques that separate dead and DNA damaged sperm from a viable sperm sample. The sperm in the dead and dying sperm population present a higher frequency of DNA fragmentation, while the sperm separated into the viable subpopulation presents reduced levels of sperm DNA fragmentation. High levels of sperm damage can be detected by incorporating various sorting techniques, such as sex sorting using flow cytometry whereby the sperm can be sorted into a dead subpopulation while another fraction may contain a live subpopulation, as for example live X- and/or live Y-chromosome bearing subpopulations. Both the dead and the live subpopulations may contain cells that are damaged or are undergoing DNA fragmentation, but the proportional distribution will be minimal in the live subpopulations of cells.

### Experiments Conducted to Demonstrate the Decrease in DNA Damage.

In the following experiments, 5 Jersey and 15 Holstein bulls were selected between the ages of 3 and 9 years of age for sperm samples. Each sex sorted sample was sorted using a MoFlo SX™ (Beckman Coulter, Miami, Fla.). Sperm was sorted based on the difference of fluorescence signals generated using Hoechst 33342 (Molecular Probes, Eugene, Oreg.) and red food dye (FD&C#40, Molecular Probes Eugene, Oreg.). The resulting fluorescent signal was stronger for the cells having a higher DNA content, i.e. X-chromosome bearing populations versus those with lower DNA content, namely Y-chromosome bearing populations. The red food dye was excluded from those sperm having healthy intact membranes, while it was retained in sperm with damaged membranes quenching a significant amount of the fluorescence signal in those cells.

In each experiment, X- and Y-chromosome bearing, sorted sperm were selected based on differences in fluorescence signals using 16.2 mM Hoechst 33342 (Molecular Probes, Eugene, Oreg., USA), diluted in catch fluid consisting of a 20% egg yolk-TRIS extender. The same standards for routine semen preparation and cut-off values for standard semen characteristics for selecting the ejaculates for processing were applied. In all experiments, the bull ejaculates for processing either conventional or sex-sorted straws of semen were used only if they met the following criteria: 1) minimum motility of 55%; 2) minimum concentration of 900×10⁶ sperm/mL, determined using the SP1-Cassette, Reagent 5100, and NucleoCounter® SP-100™ system (ChemoMetec A/S, Gydevang 43, DK-3450 Allerød, Denmark); and 3) primary morphologies 15%, secondary morphologies 15%, and a total morphology count not to exceed 25%. Further, samples used in the post-thaw analyses had to meet standard quality control conditions of: 1) progressive motility at least 45% at 0 h and 30% at 3 h; and 2) including intact acrosomes of at least 50% at 3 h. For three hour post-thaw motility and acrosome measurements, all samples were incubated for 3 h at 37° C. in a humidified chamber. For all semen quality evaluations, 75×25 mm glass microscope slides (Andwin, Addison, Ill., USA) and 22×22 mm #1.5 coverslips (Thomas Scientific, Swedesboro, N.J., USA) were used. All motility assessments were made using bright-field microscopy, and post intact acrosomes and morphology assessments were made using differential interference contrast (DIC) microscopy with a magnification of ×400.

All extenders used in the experiments were of the same formulation having a pH of 6.8 and an osmolarity balanced at 300 mOsm for the TRIS extender. For cryopreservation, sorted and conventional sperm samples were processed using a two step extension with glycerol. All frozen-thawed sex-sorted samples used in the experiments contained 2.1×10⁶ spermatozoa/straw (0.25 cc) while conventional samples had in the range of 25×10⁶ to 30×10⁶ spermatozoa/straw (0.5 cc).

Neat semen from each individual bull was divided into two aliquots. One aliquot was sex-sorted and thereafter the spermatozoa were frozen following cryostabilization using an automated freezing device, IMV Digitcool® (IMV, Cedex, France) and stored in liquid nitrogen. The second aliquot was directly cryopreserved for subsequent analysis of the level of DNA fragmentation after thawing. The sperm DNA fragmentation analysis was performed on the different subpopulations, after X- and Y-chromosome sex selection, while comparing both aliquots for each respective bull.

In each experiment DNA fragmentation was determined with a Sperm-Halomax kit (Halotech DNA, Madrid, Spain). Each sperm sample was lysed then prepared in agarose on slides. The slides were then stained with SYBR I (Invitrogen, Molecular Probes, Eugene, Oreg.) or GELRED (Biotium, Hayward, Calif.) for staining chromatin which disperses differently around lysed sperm with DNA fragmentation and those without. Cells having fragmented DNA and those with intact DNA can then be visually distinguished on each slide.

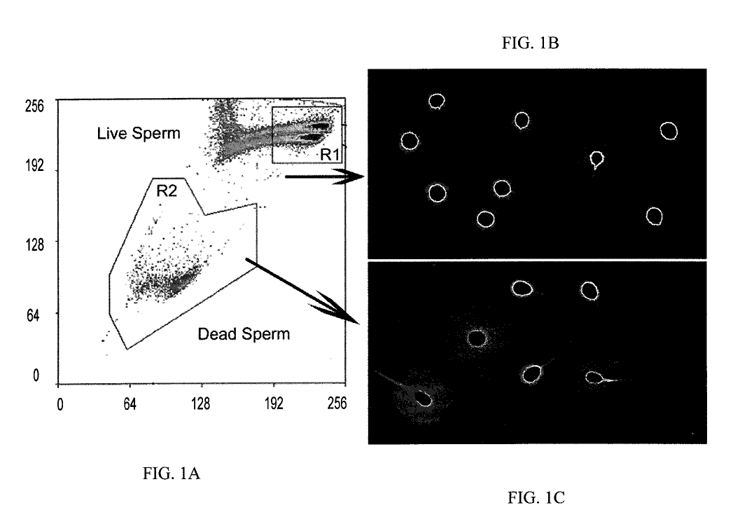

Referring to FIG. 1, a plot can be seen for sperm sorted in a flow cytometer of forward fluorescence versus side fluorescence (FIG. 1A). One subpopulation on this plot comprises a large proportion of spermatozoa which were dead or dying (R2 in FIG. 1A), and the other group comprises live spermatozoa (R1 in FIG. 1A). The regions for the sorting parameters as indicated on commercial flow cytometers such as the MoFlo SX or the MoFlo SX XDP (Beckman Coulter, Miami, Fla.) are indicated which illustrate plots for gating sperm cells based on forward and side fluorescence. FIG. 1B illustrates an in situ fluorescent micrograph of sperm cells from R1 using the Sperm-Halomax kit, in which the cells having small tight halos indicate sperm which had not undergone DNA fragmentation. In this slide, a single sperm can be seen with chromatin loosely spread around the membrane indicating this sperm had or was undergoing DNA fragmentation. Referring to FIG. 1C, sperm from R2 are illustrated, several of which can be seen with wide halos of dispersed chromatin indicating DNA fragmentation.

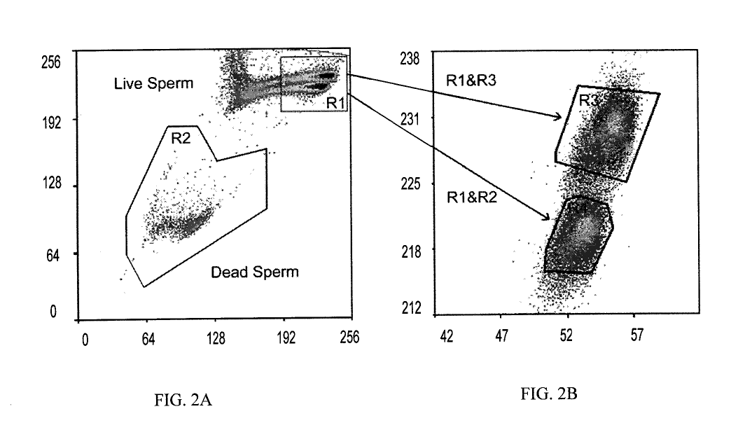

Referring to FIGS. 2A and 2B, one subpopulation included spermatozoa which were predominantly dead (R2 in FIG. 2A) and the other two groups consisted of predominantly live spermatozoa (R1 in FIG. 2A) subpopulations containing X-chromosome bearing (R3 in FIG. 2B) and Y-chromosome bearing spermatozoa (R4 in FIG. 2B) at a purity of about 95%. The MoFlo SX XDP can be configured for gating each of R3, R4, and R2 into separate containers, while the MoFlo SX can be used for separating R1 sperm from R2 sperm. Sex ratio purities of the samples were determined using an STS Sexed Semen Purity Analyzer (Sexing Technologies, Navasota, Tex.), which provides high resolution peaks of X and Y chromosome bearing spermatozoa populations and basing each analysis on 2,000 spermatozoa. All of these subpopulations were analyzed and compared for the level of DNA fragmentation relative to the level obtained in the respective pre-sort sample taken after staining and incubation but before sorting. A total of 2×10⁶ spermatozoa for each sample were sorted. Dead spermatozoa were sorted based on Region 2 in FIG. 2A, excluding all other cells falling outside that region. The proportion of dead cells in the pre-sort semen samples averaged 13%, thereby providing an average sort speed of 800 to 900 dead spermatozoa per second. Therefore, about 85% of sperm containing fragmented DNA was removed by this process from the original sample.

Experiment 1—

The first experiment was conducted to analyze the differences in the amount of DNA fragmentation before and after sex sorting. Sperm samples were taken from 5 jersey bulls and divided into two aliquots each. The first group of aliquots was sex sorted and cryopreserved. The second group of aliquots was directly cryopreserved. Table 1 illustrates the relative levels of DNA fragmentation obtained in each bull pre- and post-sex sorting.

TABLE 1
(% DNA Fragmentation, Sex Sorted)

| Reference | Pre-sort | Sort - XY |
|---|---|---|
| Bull 1 | 7.00 | 1.10 |
| Bull 2 | 7.50 | 1.10 |
| Bull 3 | 11.00 | 4.00 |
| Bull 4 | 9.00 | 5.00 |
| Bull 5 | 5.30 | 4.60 |
| Average ± SD | 7.96 ± 2.15 | 3.16 ± 1.91 |

The baseline level of DNA damage in the 5 presorted bull samples ranged from 5.3% to 11% with a mean and standard deviation of 7.9±2.1. The level of sperm DNA fragmentation obtained in sex sorted sperm samples was much lower, with a mean and standard deviation of 3.1±1.9. On average the reduction in sperm DNA fragmentation was 63%, but the reduction was as high as 85% in Bull 2.

Experiment 2—

The second experiment specifically looked at the DNA fragmentation among each sorted subpopulation after sex sorting. Again 5 jersey bulls were used for this experiment; each was collected and sorted resulting in three subpopulations of sperm. The first subpopulation of sperm primarily consisted of those sperm considered dead via conventional sorting techniques as indicated by red food dye or propidium iodine. The second and third subpopulations primarily consisted of the live sorted sperm cells. A portion of each sample was also tested prior to sorting in order to establish a baseline for DNA fragmentation. As shown in Table 2, the baseline for DNA fragmentation had a mean and standard deviation of 7.9±2.5. The DNA fragmentation determined in the sorted X-chromosome bearing subpopulation had a mean and standard deviation of 1.8±1.5, while the Y-chromosome bearing subpopulation had mean and standard deviation of 1.2±0.6 when averaged over each bull. The third subpopulation of sperm containing all the dead sperm tended to accumulate the majority of DNA fragmented sperm having a mean and standard deviation of 12±4.4.

TABLE 2
(% DNA fragmentation, sorted subpopulations)

| Reference | Pre-sort | Sort - Dead | Sort - X | Sort - Y |
|---|---|---|---|---|
| Average ± SD | 7.9 ± 2.5 | 12 ± 4.4 | 1.8 ± 1.5 | 1.2 ± 0.6 |

Experiment 3—

The third experiment was conducted to analyze the distribution of sperm DNA fragmentation in 100 sex sorted straws after thawing, for comparing variations among samples taken at different times for 10 Holstein bulls. Each straw was collected and sex sorted for X-chromosome bearing sperm. Straws collected from the same bulls on different dates tended to present very similar DNA fragmentation, as can be seen in Table 3. While there were occasional outliers, the majority of samples taken from individual bulls demonstrated similar DNA fragmentation regardless of whether they were taken on different days.

TABLE 3
(% DNA Fragmentation, X-sorted samples taken different days)
Semen Sample

| Ref. | 1 | 2 | 3 | 4 | 5 | 6 | 7 | 8 | 9 | 10 | Avg. |
|---|---|---|---|---|---|---|---|---|---|---|---|
| HO-01 | 1.00 | 0.66 | 1.00 | 0.66 | 0.66 | 0.00 | 0.33 | 1.66 | 0.66 | 0.66 | 0.73 |
| HO-02 | 0.00 | 0.00 | 0.66 | 0.33 | 0.00 | 0.30 | 0.00 | 0.66 | 0.66 | 0.50 | 0.31 |
| HO-03 | 1.00 | 0.66 | 0.33 | 0.66 | 1.00 | 1.00 | 1.33 | 0.33 | 1.00 | 1.00 | 0.83 |
| HO-04 | 0.66 | 0.33 | 0.33 | 0.33 | 0.66 | 1.00 | 0.66 | 0.66 | 0.66 | 0.00 | 0.53 |
| HO-05 | 0.33 | 0.00 | 0.30 | 0.66 | 0.00 | 2.00 | 0.30 | 0.00 | 0.33 | 1.00 | 0.49 |
| HO-06 | 0.00 | 0.00 | 0.00 | 0.00 | 0.33 | 0.00 | 0.00 | 0.00 | 0.00 | 0.00 | 0.03 |
| HO-07 | 0.00 | 0.66 | 0.33 | 0.33 | 0.66 | 0.00 | 0.00 | 0.00 | 0.00 | 0.66 | 0.26 |
| HO-08 | 0.00 | 0.00 | 0.00 | 0.00 | 0.00 | 0.00 | 0.33 | 0.00 | 0.00 | 0.00 | 0.03 |
| HO-09 | 1.66 | 0.00 | 0.33 | 1.66 | 0.33 | 0.00 | 0.00 | 0.00 | 0.00 | 0.00 | 0.40 |
| HO-10 | 0.33 | 0.66 | 0.00 | 1.66 | 0.33 | 0.33 | 0.00 | 1.00 | 0.00 | 0.00 | 0.43 |

Experiment 4—

The fourth experiment focused on evaluating DNA fragmentation in conventional and sorted samples at regular intervals in order to determine the rates at which DNA fragmentation occurs in each sample. Conventional sperm has been shown in previous experiments, and is shown again in Table 4, to have a higher baseline of DNA fragmentation as compared to sex sorted sperm. However, after monitoring sex sorted sperm at 24, 48 and 72 hours it appears sex sorted sperm is subject to a sharp increase in DNA fragmentation between about 24 and 48 hours, whereas conventional sperm maintain a baseline level until at least about 72 hours. At about 48 hours conventional sperm begin to exhibit slight increases in DNA fragmentation. Table 4 illustrates DNA fragmentation in eight bulls for conventional sperm at t0 (C-To), as well as sperm DNA fragmentation determined in sex sorted samples of the same bulls at a t0 (S-T0). As expected, S-T0 is categorically lower for each bull compared to C-T0. However, 24 hours later (S-T24), 48 hours later (S-T48) and 72 hours later (S-T72) the DNA fragmentation of the sex sorted samples change drastically, while conventional sperm tends to remain closer to its baseline level for about 72 hours. Table 4 also indicates a crossover positioning time point (CPT) which can be used as an indicator of the rate of sperm DNA fragmentation, for example, a lower CPT indicates a faster increase in DNA fragmentation and a higher CPT indicates a slower increase in DNA fragmentation. Averaged across each bull, the CPT for all 8 bulls averaged to about 33 hours. On average, after 33 hours the DNA fragmentation became greater in the sex sorted samples than in the conventional samples.

TABLE 4
(CPT and % DNA Fragmentation - Sorted Over Time)

| | CPT | C-T0 | S-T0 | S-T24 | S-T48 | S-T72 |
|---|---|---|---|---|---|---|
| Bull 1 | 27 h | 5.00 | 0.33 | 1.66 | 10.50 | 70.00 |
| Bull 2 | 44 h | 2.00 | 0.33 | 0.66 | 5.00 | 45.00 |
| Bull 3 | 25 h | 2.66 | 0.33 | 0.66 | 17.00 | 32.00 |
| Bull 4 | 33 h | 2.00 | 0.33 | 0.00 | 11.00 | 26.00 |
| Bull 5 | 51 h | 4.00 | 0.00 | 0.66 | 2.50 | 66.00 |
| Bull 6 | 41 h | 4.00 | 2.00 | 2.66 | 8.00 | 29.00 |
| Bull 7 | 27 h | 3.33 | 0.00 | 1.00 | 19.66 | 32.00 |
| Bull 8 | 19 h | 1.00 | 0.00 | 1.00 | 19.00 | 37.00 |
| r2 | | 0.31 | 0.27 | 0.59 | 0.64 | 0.68 |
| Durbin- | | 2.05 | 1.98 | 1.99 | 2.41 | 2.55 |
| Watson | | | | | | |
| P | | 0.24 | 0.26 | 0.27 | 0.09 | 0.06 |

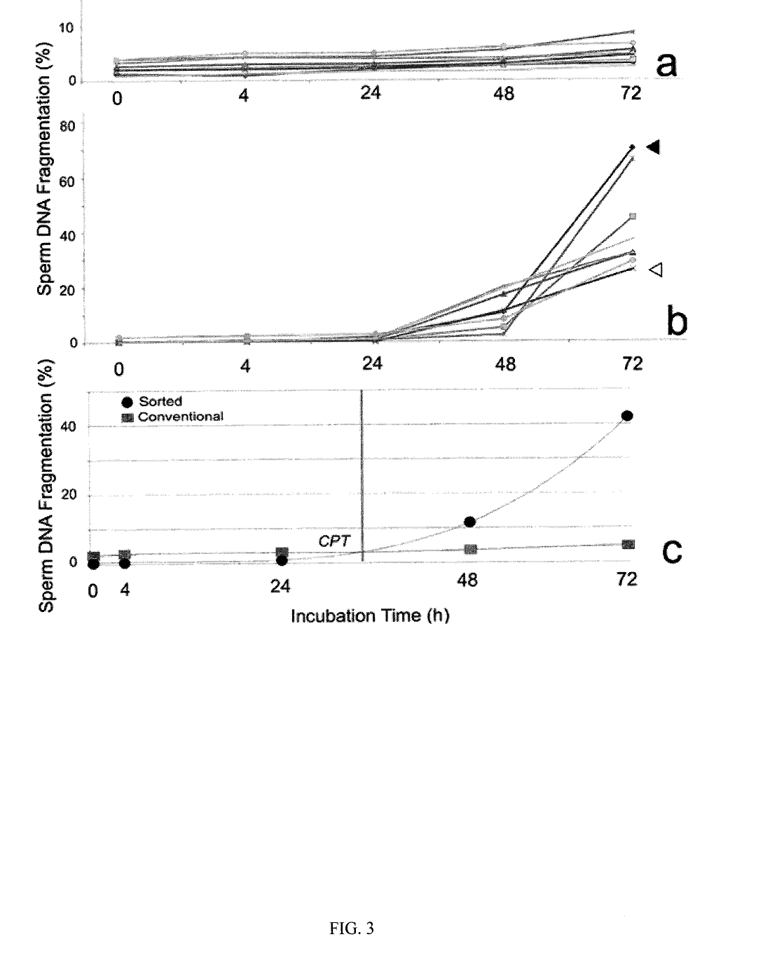

FIG. 3 illustrates a graphical representation of the data in Table 4. FIG. 3A illustrates the percentage of DNA fragmentation over time for about 72 hours. In comparison, FIG. 3B illustrates the DNA fragmentation in sorted sperm over the course of 72 hours. Contrasting FIG. 3A with FIG. 3B is it can be seen the conventional sperm increases at a slower and more steady rate over 72 hours of incubation, while the sorted sperm often presents sharp increases in sperm DNA fragmentation between about 24 and 48 hours. FIG. 3C illustrates the mean the conventional samples and the mean of the sorted samples and by doing so more clearly demonstrates the sorted sperm having lower percentages of DNA fragmentation initially. FIG. 3C also more clearly illustrates the sharp increase DNA fragmentation among the sorted sperm relative to the conventional sperm and the CPT, where on average the sorted sperm began presenting more DNA fragmentation than the sorted sperm. For the samples taken and illustrated in FIG. 3C the CPT occurs around 33 hours.

Enriching a Population of Sperm with Respect to DNA Fragmentation.

In one embodiment, once a sperm sample has been stained with a molecular marker, the sperm can be examined by flow cytometry. It should be appreciated that other methods for measuring and detecting molecular markers and fluorescent markers can be used including, but not limited to, the use of a spectrophotometer and microfluidic chips and these should be considered embodiments of the disclosed methods. Flow cytometry can be used as described in U.S. Pat. No. 6,357,307, as referenced above, to determine the amount of DNA in each cell, and the cells can be separated based on this measurement. As DNA fragmentation occurs within a given cell, the number of binding cites for a DNA selective dye can be reduced depending upon the location of the fragmentation sites. This means sperm undergoing DNA fragmentation might tend to have less of these binding cites available for stoichiometrically binding fluorescent dyes, such as Hoechst 33342. Similarly, flow cytometry can be used to differentiate cells with broken or altered membranes from viable cells based on the fluorescence measurements with a second marker, such as propidium iodide or red food dye.

Once the cells have been evaluated, they can be separated in a number of ways. U.S. Pat. No. 6,357,307 discusses the use of electromagnetic deflection used in conjunction with flow cytometry. However, embodiments of the current method also contemplate the use of microfluidic channels for separating the cells. US Patent Application Publication 2006/0270021 and U.S. Pat. No. 7,298,478, the entire contents of each incorporated herein by reference, provide examples of microfluidic channels which could be used for the separation of sperm.

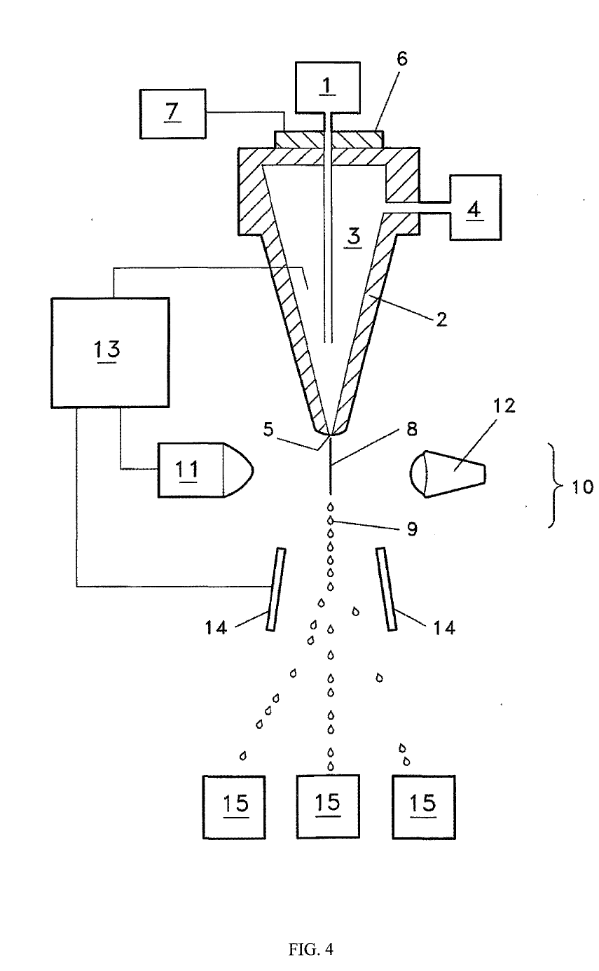

Turning now to FIG. 4, a system is illustrated for carrying out certain methods described herein. The system includes a cell source 1 for establishing a supply of cells for analysis and/or sorting. The cells are introduced into a nozzle 2 along with a sheath fluid 3 is introduced from a sheath fluid source 4. The sheath fluid 3 forms a sheath fluid environment around the cells as both are fed out of the nozzle 2 through a nozzle orifice 5.

The pressure with which fluids are supplied to the nozzle 2 affects the velocity of a stream 8 exiting the nozzle orifice 5. The stream 8 can further be controlled by an oscillator 6 through an oscillator controller 7 which produces pressure waves in nozzle 2 and the nozzle orifice 5. These pressure waves are transferred through the nozzle 4 and nozzle orifice 5 to the stream 8, resulting in the regular formation of droplets 9 at a break off point. The diameter of the nozzle orifice 5 and the frequency of the oscillator 6 can be coordinated to produce droplets which are large enough to entrain isolated cells.

The droplets 9 entraining individual cells can be analyzed and/or sorted based on the characteristics of the cells entrained in each of the droplets 9. As part of the flow cytometer, a cell sensing system 10 can be incorporated for making these distinctions. The cell sensing system 10 can include a detector or sensor 11 which responds to the cells contained within the stream 8. The cell sensing system 10 can cause an action depending on the relative presence or absence of a characteristic. For example, the presence, absence or quantity of a marker molecule can be used in order to characterize cells as more likely to be undergoing DNA fragmentation from those which are less likely to be undergoing sperm DNA fragmentation.

As one example, a fluorochrome dye can be bound to the DNA within the cell as a molecular marker. The fluorochrome dye can be excited by an excitation device 12, such as a laser, which emits an irradiation beam causing the fluorochrome dye to react or fluoresce. For the purpose of sex sorting sperm, each sperm is subjected to staining by a DNA binding fluorescent dye, such as Hoechst 33342. The total fluorescence of each passing cell is dependent upon the amount of DNA contained within each cell, thereby providing a means for distinguishing X chromosome bearing sperm from Y chromosome bearing sperm. As previously described these emissions can also provide information relating to the DNA fragmentation characteristics of individual cells.

The fluorescence can be picked up by a sensor 11 and converted into an electrical signal. That electrical signal can be input into an analyzer 13 for making a determination based on the emitted fluorescence. The analyzer 13 can be coupled to a droplet charger for differentially charging the stream 8, and thus droplets 9 just prior to their break off. The timing of the detection and the charging is coordinated such that the stream is charged just prior to the break off of a droplet in containing the analyzed cell. Once the droplet is broken off, it retains the charge of the stream.

FIG. 4 also illustrates deflection plates 14 on either side of the nozzle 2 in order to direct cells into one of several possible trajectories. The deflection plates can be charged with opposite electrical fields, for example approximately +2500 Volts for the left hand plate and −2500 Volts right hand plate respectively. It should be appreciated that depending on the apparatus, the plates can be charged up to about 4000 Volts in either polarization. Those in the field familiar with the operation of flow cytometers can set up such deflection plates in any number of configurations using various charge configurations. For example, the faster the velocity of the flow stream the more voltage is required to pull a droplet onto a specific trajectory. As droplets fall between the deflection plates, they will be attracted towards the plate having an opposite charge, or fall straight downwards in the case where no charge is applied to a droplet 9. The collection containers 15 are illustrated as three containers for collecting droplets which: have not been charged, which have been positively charged, and which have been negatively charged. It should be appreciated that the arrangement of three containers 15 illustrated in FIG. 4 can be configured on the MoFlo SX, but that one of the illustrated streams is for waste, or empty droplets. Therefore, in order to sort a sample into three populations, an additional container is required for waste. The MoFlo SX XDP can be configured with two streams, such as an X-enriched and a Y-enriched stream, in addition to a dead cell stream and a waste stream. This machine can also be gated in order to produce a single sorted sample having about 50/50 of each sex, but being enriched for DNA fragmentation characteristics.

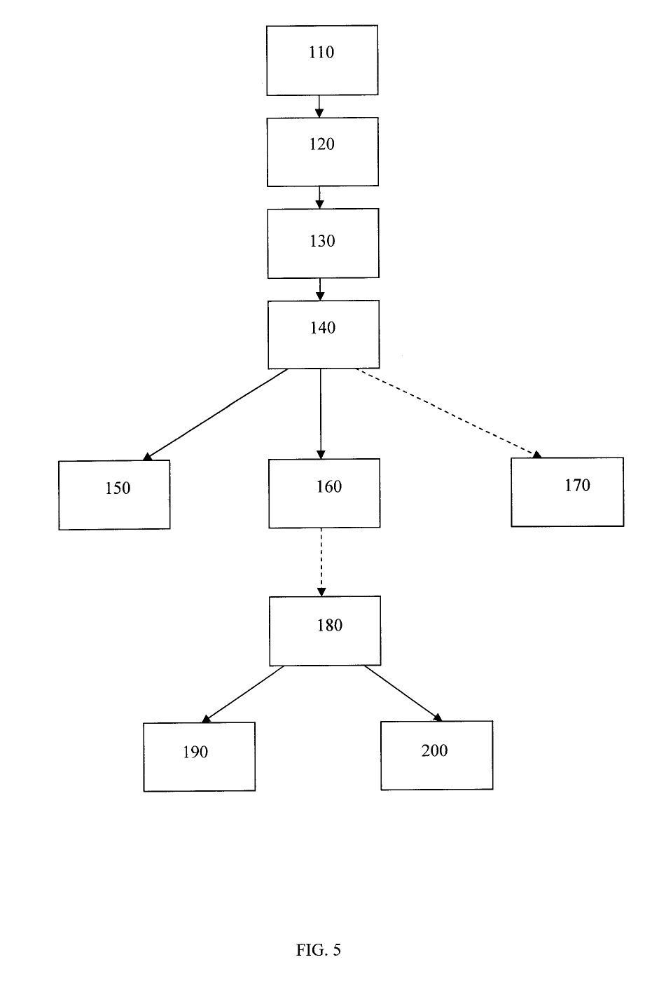

Turning now to FIG. 5, a flow chart illustrates the step of establishing a fluid sample at 110 for sorting. This fluid sample can include semen or another inseminant containing sperm or sex sorted sperm, or another form or processed sperm. Once the sample is established, the flow chart proceeds to the step marking the sample with a molecular marker 120. Marking can include staining and the molecular marker can be a fluorescent dye, a non-fluorescent dye, antibodies, propidium iodide, a fluorophore, or a fluorophore-like substance, including the dyes previously discussed.

Once the sample is marked, the cells in the sample are evaluated at 130. Flow cytometry, spectrometry, or other methods, depending on the molecular markers employed, can be used to evaluate the molecular makers for determining DNA fragmentation characteristics, the amount of DNA present in a cell, fractures in DNA, and/or compromised cell membranes.

Once this evaluation is made, the cells can be separated at 140. This separation can be accomplished in flow cytometry through the use of electromagnetic deflection, and in the alternative microfluidic chambers can be used to separate the reproductive cells. Other cell separation techniques can also be used. The cells can be separated on the basis of carrying an X- or Y-chromosome, as well as, the presence or absence of markers. Or, the cells can be sorted into a single population of sperm having both X- and Y-chromosomes excluding cells with higher incidences of DNA fragmentation.

Step 150 represents the formation of a first subset of cells formed by separation of step 140. The first subset of cells can be selected to have a higher percentage of DNA fragmentation, or apoptosis as compared to the original sample. In one embodiment, the first subset of cells has a higher percentage of cells with DNA fragmentation as compared to the original fluid sample. This subset can be selected for having low fluorescence emissions, or relatively lower as compared to the general population of cells. Step 160 represents the formation of a second subset of cells. The second subset can include either cells sorted for the X-chromosome, cells sorted for the Y-chromosome, or indiscriminately cells with either the X- or the Y-chromosome. Step 170 represents an optional embodiment where a third subset of cells is formed. The third subset of sperm can be selected as a complimentary gender subset as compared to the second. Each of the second subset and the third subset of sperm can be selected based on unique levels of DNA fragmentation or cellular characteristics. As an example, the first subset can include sperm having DNA fragmentation characteristics indicating a higher percentage of DNA fragmentation, while the second subset, and optionally the third subset, can have DNA fragmentation characteristics indicating reduced levels of DNA fragmentation.

By way of a non-limiting example, the three subsets can represent the separation of sperm demonstrating a higher level of DNA fragmentation in the first subset 150, viable sperm carrying an X-chromosome in the second subset 160 and viable sperm carrying a Y-chromosome in the third subset. Each of the second and third subsets is selected from sperm which demonstrate minimal levels of DNA fragmentation characteristics. Other embodiments of the method contemplate more than three subsets which can be selected based on measured values of one or more molecular markers. For example, the subsets can be selected based on expected purity differences.

By way of another non limiting example, the first subset 150 can contain a subpopulation of sperm determined not to be viable, or having a high likelihood of not being viable, while the second subset includes sperm which is determined to be viable, or have a higher likelihood of being viable.

Step 180 represents a new measurement/separation that can occur subsequent to the second subset 160 or concurrently with the measurements and separations of steps 130 and 140.

By way of a non-limiting example, the second subset 160 can be sperm determined in step 130 to be viable and separated into a second subset 160 in step 140. This subset of sperm 160 can then be sex sorted by flow cytometry in step 180 into a group of sperm carrying X-chromosomes 190 and a group of sperm carrying Y-chromosomes 200.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A method for sorting cells comprising the steps of:
a. establishing a sperm sample;
b. applying a molecular marker to the sperm sample;
c. exciting sperm individually within the sperm sample;
d. monitoring the excited sperm;
e. evaluating the sperm for DNA fragmentation characteristics; and
f. selecting a population enriched for non-fragmented sperm.

2. The method according to claim 1 wherein the step of separating the sperm further comprises the step of sorting the sperm sample into at least a first population, a second population and a third population, wherein the first population contains a greater percentage of cells with DNA fragmentation as compared to the original sperm sample, as well a greater percentage of cells with DNA fragmentation as compared to the second population and the third population.

3. The method according to claim 1 wherein the step of separating the sperm further comprises the step of sorting the sperm sample into a first population having sperm with a greater incidence of DNA fragmentation and a second population having a lower incidence of DNA fragmentation.

4. The method according to claim 3 wherein the first population comprises about a 50/50 mixture of sperm bearing X-chromosomes and sperm bearing Y-chromosomes.

5. The method of claim 1 wherein the marker comprises a fluorochrome dye with which the sperm sample is stained.

6. The method of claim 5 wherein the stained sperm are excited with a laser.

7. The method of claim 6 wherein the step of monitoring sperm for DNA fragmentation further comprises detecting fluorescence emitted from the excited cells.

8. The method of claim 7 wherein the step of evaluating the sperm for DNA fragmentation further comprises differentiating DNA fragmentation characteristics based on the emitted fluorescence of each of the cells.

9. The method according to claim 1 further comprising the step of staining the sperm sample with a second marker.

10. The method of claim 8 wherein the second marker comprises red food dye or propidium iodide.

11. The method of claim 1 wherein the step of separating the cells or groups of cells results in at least a first population of sperm and a second population of sperm, wherein the first population of cells has a greater percentage of cells with DNA fragmentation as compared to the original sample and as compared to the second population of cells.

12. The method of claim 11 wherein the first population of sperm comprises cells which are regarded as dead cells and wherein the second population of sperm are regarded as viable sperm.

13. The method of claim 1 wherein the step of evaluating the cells or groups of cells for DNA fragmentation includes the step of performing a flow cytometry, spectrometry, or a microfluidic analysis of the fluid sample.

14. The method of claim 1 wherein the sperm sample comprises mammalian sperm.

15. The method of claim 1 wherein a sperm sample selected for having less DNA fragmentation provides improved fertility characteristics.

16. The method according to claim 15 wherein the improved fertility characteristics are selected from: cleavage rates, blastocysts rates, pregnancy rates, and birth rates.

17. A flow cytometer system for sorting cells comprising:
a. an inlet for receiving a sperm sample;
b. one or a combination of molecular markers capable of discriminating the level of DNA fragmentation in a sperm;
c. an outlet for producing droplets entraining sperm from the sperm sample;
d. an excitation device positioned to produce an irradiation beam through the stream at an inspection zone;
e. a detector positioned to detect the interaction of the radiant energy with the marker associated with the DNA of sperm at the inspection zone and to produce a signal characterizing the detected interaction;
f. an analyzer connected to the detector for analyzing the produced signal;
g. a separator connected to the analyzer for separating the sample into distinct sorted sperm populations based on the signal produced.

18. A flow cytometer system described in claim 17 further comprising a second marker.

19. The flow cytometer of claim 18 wherein the second marker comprises red food dye or propidium iodide.

20. The flow cytometer system described in claim 17 further comprising:
a. a first collection element for collecting a first population of sorted sperm; and
b. a second collection element for collecting a second population of sperm.

21. The flow cytometer system described in claim 20 further comprising a third collection element for collecting a third population of sperm.

22. The flow cytometer system of claim 20 wherein the first population of sperm has less DNA fragmentation than the sperm in the sample.

23. The flow cytometer system of claim 17 wherein the marker comprises one or more components selected from the group consisting of: antibody, fluorescent dye, and non-fluorescent dye.

24. The flow cytometer system of claim 23 wherein the fluorescent dye comprises fluorochrome or a fluorophore.

25. A method of improving the fertility in a sperm sample comprising:
a. establishing a sperm sample;
b. applying a molecular marker to the sperm sample;
c. exciting sperm individually within the sperm sample;
d. monitoring the excited sperm;
e. evaluating the sperm for DNA fragmentation characteristics; and
f. selecting a population enriched for non-fragmented sperm and improved fertility.

26. A method of improving the fertility of an assisted reproductive procedure comprising:
a. obtaining a sperm sample with improved fertility according to claim 25; and
b. fertilizing an egg.

27. The method of improving the fertility of an assisted reproductive procedure of claim 26, wherein the assisted reproductive procedure is selected from the group consisting of: artificial insemination, in vitro fertilization and intracytoplamic injection.
